SEQUENCE LISTING (6 sequences)

SEQ ID NO: 1 - protein, 294 aa, Mycobacterium tuberculosis
SRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 2 - protein, 284 aa, Mycobacterium tuberculosis
SRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAANMWGPSSDPAWERNDPTQQIPKLVANNTRLWVYCGNGTPNELGGANIPAEFLKNFVRSSNLKFQDAYNAAGGHNAVFNFPPNGTHSWEYWGAQLNAMKGDLQSSLGAG

SEQ ID NO: 3 - protein, 294 aa, Mycobacterium tuberculosis
FSRPGLPVEYLQVPSASMGRDIKVQFQGGGPHAVYLLDGLRAQDDYNGWDINTPAFEEYYQSGLSVIMPVGGQSSFYTDWYQPSQSNGQNYTYKWETFLTREMPAWLQANKGVSPTGNAAVGLSMSGGSALILAAYYPQQFPYAASLSGFLNPSEGWWPTLIGLAMNDSGGYNANSMWGPSSDPAWKRNDPMVQIPRLVANNTRIWVYCGNGTPSDLGGDNIPAKFLEGLTLRTNQTFRDTYAADGGRNGVFNFPPNGTHSWPYWNEQLVAMKADIQHVLNGATPPAAPAAPAA

SEQ ID NO: 4 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 5 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAANMWGPSSDPAWERNDPTQQIPKLVANNTRLWVYCGNGTPNELGGANIPAEFLKNFVRSSNLKFQDAYNAAGGHNAVFNFPPNGTHSWEYWGAQLNAMKGDLQSSLGAG

SEQ ID NO: 6 - protein, 340 aa, Mycobacterium tuberculosis
MTFFEQVRRLRSAATTLPRRLAIAAMGAVLVYGLVGTFGGPATAGAFSRPGLPVEYLQVPSASMGRDIKVQFQGGGPHAVYLLDGLRAQDDYNGWDINTPAFEEYYQSGLSVIMPVGGQSSFYTDWYQPSQSNGQNYTYKWETFLTREMPAWLQANKGVSPTGNAAVGLSMSGGSALILAAYYPQQFPYAASLSGFLNPSEGWWPTLIGLAMNDSGGYNANSMWGPSSDPAWKRNDPMVQIPRLVANNTRIWVYCGNGTPSDLGGDNIPAKFLEGLTLRTNQTFRDTYAADGGRNGVFNFPPNGTHSWPYWNEQLVAMKADIQHVLNGATPPAAPAAPAA

(19) United States
(12) Patent Application Publication
(10) Pub. No.: US 2023/0008380 A1
(43) Pub. Date: Jan. 12, 2023

(54) M. TUBERCULOSIS AG85 PROTEINS AND METHODS OF USE

(71) Applicant: UNIVERSITY OF GEORGIA RESEARCH FOUNDATION, INC., Athens, GA (US)

(72) Inventors: Ahmet OZDILEK, Athens, GA (US); Fikri Y. AVCI, Athens, GA (US)

(73) Assignee: UNIVERSITY OF GEORGIA RESEARCH FOUNDATION, INC., Athens, GA (US)

(21) Appl. No.: 17/783,379

(22) PCT Filed: Dec. 8, 2020
(86) PCT No.: PCT/US2020/063744

§ 371 Date: Jun. 8, 2022

Related U.S. Application Data
(60) Provisional application No. 62/945,626, filed on Dec. 9, 2019.

(51) Int. Cl. A61K 39/04; C07K 14/35; A61P 31/06

(52) U.S. Cl. CPC A61K 39/04; C07K 14/35; A61P 31/06; A61K 2039/53

(57) ABSTRACT

The present disclosure provides isolated polynucleotides encoding a mature Mycobacterium tuberculosis protein selected from Ag85A, Ag85B, and Ag85C, where the protein does not include a signal for glycosylation, such as a N-glycosylation consensus sequon. Also disclosed are M. tuberculosis proteins selected from Ag85A, Ag85B, and Ag85C that do not include a signal for glycosylation, such as a N-glycosylation consensus sequon, and/or are not glycosylated, and methods for using the polynucleotides and proteins.

### CROSS-REFERENCE TO RELATED APPLICATIONS

This application claims the benefit of U.S. Provisional Application Ser. No. 62/945,626, filed Dec. 9, 2019, which is incorporated by reference herein in its entirety.

### GOVERNMENT FUNDING

This invention was made with government support under R01AI123383 awarded by National Institutes of Health. The government has certain rights in the invention.

### BACKGROUND

The use of nucleic acid vaccines, e.g., DNA vaccines, recombinant vector vaccines, and messenger RNA vaccines, is a new technology for immunizing individuals (1). This next-generation vaccine approach adopts a different modus operandi than other vaccine types in use today: rather than using whole pathogens or purified protein products, these vaccines introduce the genetic materials encoding antigenic, pathogen-associated proteins into the host cells. Unlike viral proteins, proteins of nonviral pathogens are not expressed in host cells upon infection, thus proteins of nonviral pathogens are expressed in a different system (e.g., eukaryotic versus prokaryotic systems).

Nucleic acid vaccines were produced to express in human cells the Mycobacterium tuberculosis (Mtb) surface protein Ag85A, a target protein used in multiple clinical vaccine trials (Voss et al., 2018, F1000 Res. 7, 199). In three different clinical trials, however, the nucleic acid-based Ag85A vaccine candidates were not protective or immunogenic among the participants (Tameris et al., 2013, Lancet 381, 1021-1028; Ndiaye et al.; 2015, Lancet Respir. Med. 3, 190-200; Tameris et al., 2015, Vaccine 33, 2944-2954).

### SUMMARY OF THE APPLICATION

We postulated that, when nucleic acid vaccines are used for nonviral pathogens, the proteins expressed by host cells would be structurally and immunogenically different from native pathogen protein, due to the elaboration of host posttranslational modifications (PTMs), especially glycosylation for proteins of nonviral pathogens that enter the secretory pathway. Glycans may hinder or alter important epitopes and/or down-regulate immune responses through many regulatory immune cell receptors (2). Therefore, the glycans on the proteins expressed through nucleic acid vaccines may undesirably influence the immune responses against the antigen. As described herein, we investigated the impact of host glycosylation on the immunological properties of the Mtb protein Ag85A, and show that, when Ag85A is expressed in mammalian cells, it is glycosylated, does not induce a strong humoral immune response in mice, and does not activate Ag85A-specific lymphocytes as highly as Ag85A natively expressed by the bacterium.

Provided herein are isolated proteins. In one embodiment, the present disclosure includes an isolated protein having immunogenic activity, wherein the protein includes an amino acid sequence, wherein the amino acid sequence and the amino acid sequence of SEQ ID NO:1, 2, or 3 have at least 80% identity, wherein at least one amino acid of any N-glycosylation consensus sequon present in the amino acid sequence of the protein includes at least one substitution mutation, and wherein the protein is not glycosylated.

In one embodiment, the present disclosure includes an isolated protein having immunogenic activity, wherein the protein includes an amino acid sequence, wherein the amino acid sequence and the amino acid sequence of SEQ ID NO:1 have at least 80% identity, wherein at least one amino acid of the N-linked glycosylation site NNT at positions 202-204 includes at least one substitution mutation, and wherein the protein is not glycosylated. The amino acid at position 202 can be substituted, for instance to a Q.

In one embodiment, the present disclosure includes an isolated protein having immunogenic activity, wherein the protein includes an amino acid sequence, wherein the amino acid sequence and the amino acid sequence of SEQ ID NO:2 have at least 80% identity, wherein at least one amino acid of each of the N-linked glycosylation sites NNS at positions 31-33, NNT at positions 203-205, NGT at positions 213-215, and NGT at positions 259-261 includes at least one substitution mutation, and wherein the protein is not glycosylated. The amino acids at positions 31, 203, 213, and 259 can be substituted.

In one embodiment, the present disclosure includes an isolated protein having immunogenic activity, wherein the protein includes an amino acid sequence, wherein the amino acid sequence and the amino acid sequence of SEQ ID NO:3 have at least 80% identity, wherein at least one amino acid of each of the N-linked glycosylation sites NYT at positions 90-92, NDS at positions 167-169, NNT at positions 201-203, NGT at positions 211-213, NQT at positions 235-237, and NGT at positions 257-259 includes at least one substitution mutation, and wherein the protein is not glycosylated. The protein of claim 7 wherein the amino acids at positions 91, 167, 201, 211, 235, and 257 can be substituted.

In one embodiment, the present disclosure includes an isolated polynucleotide including: (a) a nucleotide sequence encoding the protein described herein, or (b) the full complement of the nucleotide sequence of (a). The polynucleotide can further include an operably linked heterologous regulatory sequence. The polynucleotide can be present in a vector.

In one embodiment, the present disclosure includes a genetically modified eukaryotic cell including an exogenous polynucleotide described herein. In one embodiment, the genetically modified cell can be a mammalian cell.

In one embodiment, the present disclosure includes a composition that includes a protein described herein, an optional pharmaceutically acceptable carrier, and/or an optional adjuvant. In one embodiment, the present disclosure includes a polynucleotide described herein, an optional pharmaceutically acceptable carrier, and/or an optional adjuvant.

In one embodiment, the present disclosure includes a method for inducing an immune response including administering to a subject an amount of a composition described herein effective to induce the subject to produce an immune response to the protein encoded by the polynucleotide.

In one embodiment, the present disclosure includes a method for treating an infection in a subject, the method including administering an effective amount of a composition described herein to a subject having or at risk of having an infection caused by Mycobacterium tuberculosis.

### Definitions

Terms used herein will be understood to take on their ordinary meaning in the relevant art unless specified otherwise. Several terms used herein, and their meanings are set forth below.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, and polypeptide are all included within the definition of protein and these terms are used interchangeably.

As used herein, an “isolated” compound, such as a protein or polynucleotide, is one that has been removed from its natural environment. A protein described herein can be produced by a cell that does not naturally produce the protein. A protein produced by such a cell is considered “isolated” when it is removed from most of the other cellular components, e.g., carbohydrates, lipids, and other proteins, that are part of the cell. Proteins and polynucleotides that are produced by recombinant, enzymatic, or chemical techniques are considered to be isolated and purified by definition, since they were never present in a natural environment.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

The terms “coding sequence” and “coding region” are used interchangeably herein, and with respect to a DNA molecule refer to a polynucleotide that encodes an RNA and, when placed under the control of appropriate regulatory sequences, expresses the encoded RNA. An mRNA can be translated in the host cell to yield a protein. With respect to a mRNA molecule, the terms “coding sequence” and “coding region” are used interchangeably herein and refer to a polynucleotide that encodes a protein. The boundaries of a coding region are generally determined by a translation start codon at its 5′ end and a translation stop codon at its 3′ end. A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

As used herein, the term “vaccine composition” and “vaccine” are used interchangeably and refer to a composition containing an antigen or containing a polynucleotide that encodes an antigen, where the composition can be used induce an immune response, including, but not limited to, preventing or treating a disease or condition in a subject.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Conditions that are “suitable” for an event to occur, such as production of an Ag85 protein, or “suitable” conditions are conditions that do not prevent such events from occurring. Thus, these conditions permit, enhance, facilitate, and/or are conducive to the event.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows the amino acid sequences of an Ag85A (SEQ ID NOs:1 and 4), an Ag85B (SEQ ID NOs:2 and 5), and an Ag85C protein (SEQ ID NOs:3 and 6). The amino acids depicted in bold and underlined are N-glycosylation consensus sequons.

FIG. 2 shows Ag85A is glycosylated when expressed in mammalian cells. (FIG. 2A) SDS-PAGE analysis of purified native/recombinant Ag85A molecules with or without PNGase F treatment. (FIG. 2B) Western blot of proteins in (FIG. 2A) probed with sera from mice immunized with purified 293-F Ag85A. (FIG. 2C) Lectin blot of proteins in (FIG. 2A) probed with concanavalin A. (FIG. 2D) Dot blot of native Ag85A and 293-F Ag85A with Sambucus nigra lectin (SNA). (FIG. 2E) Monosaccharide analysis of native Ag85A and purified 293-F Ag85A glycans by acid hydrolysis followed by HPAEC-PAD. (FIG. 2F) N-glycan analysis of 293-F Ag85A. N-glycans were released enzymatically, permethylated, and analyzed by NSI-MSn. The relative abundances of all individual glycans detected at quantifiable levels are presented as the percent of the total glycan profile contributed by each indicated glycan.

FIG. 3 shows Ag85A elicits weaker immune responses than native Ag85A. Serum IgG reactivities of mice immunized with adjuvant alone, native Ag85A or 293-F Ag85A against: native Ag85A (FIG. 3A), 293-F Ag85A (FIG. 3B), or gamma irradiated Mtb (FIG. 3C) determined by ELISA. Serum IgG titers were reported as the reciprocal dilution that results in an OD of 0.5 at 450 nm. (FIG. 3D) Intact Mtb binding of immune sera analyzed by flow cytometry. Overlaid histograms represent data from four technical repeats. (FIG. 3E) Flow cytometry analysis for opsonophagocytic activities of immune sera co-incubated with FITC-labeled Mtb and J774 mouse macrophages. Splenic mononuclear cells harvested from mice immunized with native Ag85A (FIG. 3F) or 293-F Ag85A (FIG. 3G) were assessed for their IL-2 production upon stimulation with native, 293-F or N246Q Ag85A by ELISA. IL-2 concentrations were normalized to media. (FIG. 3H) Serum IgG reactivities of mice immunized with native Ag85A against: native, 293-F or N246Q Ag85A determined by dot blot assay. Triplicate dots for each group were quantified with ImageJ software. Dot blots are obtained from the same membrane with same exposure time. Statistical significance was determined with the two-tailed Student's t test. ****, P<0.0001; ***, P<0.001; **, P<0.01; ns, not significant.

### DETAILED DESCRIPTION

### Proteins

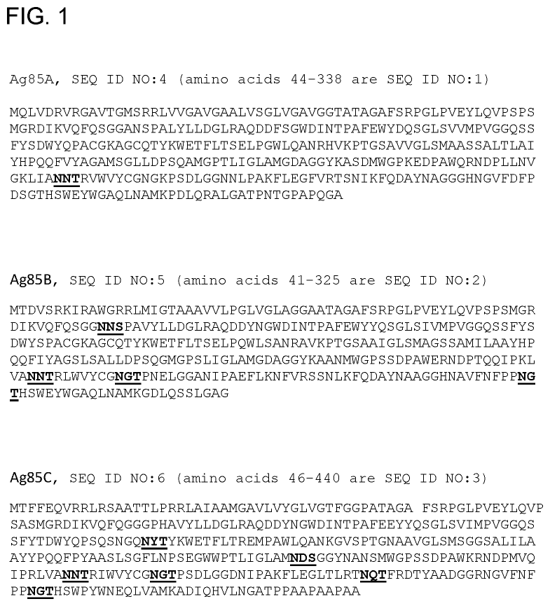

The present disclosure describes isolated proteins which are a variant of a mature Mycobacterium tuberculosis (Mtb) Ag85 protein. As used herein, a “mature” Ag85 protein is an Ag85 protein that has been processed to not include a signal sequence. In one embodiment, an Ag85 protein is a variant of a mature Ag85A (also referred to as diacylglycerol acyltransferase, and mycolyltransferase Ag85A). An example of an Ag85A protein is available at Genbank accession number P9WQP3 (SEQ ID NO:3), is shown in FIG. 1, and the mature form is amino acids 44-338 (SEQ ID NO:1). In one embodiment, an Ag85 protein is a variant of a mature Ag85B (also referred to as secreted antigen Ag85B). An example of an Ag85B protein is available at Genbank accession number Q847N4 (SEQ ID NO:4), is shown in FIG. 1, and the mature form is amino acids 41-325 (SEQ ID NO:2). In one embodiment, an Ag85 protein is a variant of a mature Ag85C (also referred to as diacylglycerol acyltransferase/mycolyltransferase Ag85C). An example of an Ag85C protein is available at Genbank accession number P9WQN8 (SEQ ID NO:6), is shown in FIG. 1, and the mature form is amino acids 46-440 (SEQ ID NO:1). As used herein, the terms “Ag85,” “Ag85A,” “Ag85B,” and “Ag85C” refer to a variant of a mature Ag85A, Ag85B, or Ag85C as described herein, unless the context indicates the wild-type protein.

Wild-type Ag85 proteins are naturally expressed by Mycobacterium tuberculosis, and wild-type Ag85 proteins are not known to be glycosylated. Wild-type Ag85 proteins are immunogenic and useful in vaccines. The inventors have determined that expression of an Ag85 protein in eukaryotic cell results in glycosylation of the protein at N-glycosylation consensus sequons, and that glycosylation causes an unexpected and significant reduction of immunogenicity. As a result, an immune response to an Ag85 protein delivered by a nucleic acid vaccine encoding wild-type Ag85 is reduced, and protective efficacy of the nucleic acid vaccine is likewise reduced.

The Ag85 proteins disclosed herein are different from the wild-type version of the protein because they include a substitution mutation of at least one amino acid. Accordingly, an Ag85 protein disclosed herein is a non-natural protein. Moreover, delivery of a protein described herein by a nucleic acid vaccine results in a significant immune response.

In one embodiment, an Ag85 protein does not include a N-glycosylation consensus sequon. For instance, an Ag85 protein can include a substitution mutation of one or more amino acids of an N-glycosylation consensus sequon, where the substitution results in either reduced or complete loss of glycosylation at that sequon when the Ag85 is expressed in a eukaryotic cell. N-glycosylation consensus sequons have the structure asparagine-X-serine/threonine (N-X-S/T), where X is any amino acid except proline (P). The substitution can be at position 1 (the N is replaced with a different amino acid), at position 2 (the amino acid at X is replaced with a proline), or at position 3 (the S or T is replaced with an amino acid that is not S or T). At position 1 any amino acid other than N can be used, and in one embodiment the amino acid Q or A can be used to replace the N. At position 3 any amino acid other than S or T can be used, and in one embodiment the amino acid A can be used to replace the S or T.

In the wild-type Ag85 proteins disclosed herein all N-glycosylated consensus sequons are identified, and a polynucleotide encoding a variant Ag85 protein is produced using standard and routine methods of molecular biology. The skilled person will recognize that N-glycosylation consensus sequons can be present in other wild-type Ag85 proteins. The example of an Ag85A protein shown in FIG. 1 (SEQ ID NO:1) includes an N-linked glycosylation site NNT at positions 202-204. An Ag85A protein includes a substitution mutation of one or more amino acids of the N-glycosylation consensus sequon present in this example of an Ag85A protein. In one embodiment, an Ag85A protein includes a substitution mutation of one or more amino acids of all N-glycosylation consensus sequons present in the Ag85A protein.

The example of an Ag85B protein shown in FIG. 1 (SEQ ID NO:2) includes N-linked glycosylation sites NNS at positions 31-33, NNT at positions 203-205, NGT at positions 213-215, and NGT at positions 259-261. An Ag85B protein includes a substitution mutation of one or more amino acids of 1, 2, 3, or all 4 of the N-glycosylation consensus sequons present in this example of an Ag85B protein. In one embodiment, an Ag85B protein includes a substitution mutation of one or more amino acids of all N-glycosylation consensus sequons present in the Ag85B protein.

The example of an Ag85C protein shown in FIG. 1 (SEQ ID NO:3) includes N-linked glycosylation sites NYT at positions 90-92, NDS at positions 167-169, NNT at positions 201-203, NGT at positions 211-213, NQT at positions 235-237, and NGT at positions 257-259. An Ag85B protein includes a substitution mutation of one or more amino acids of 1, 2, 3, 4, 5, or all 6 of the N-glycosylation consensus sequons present in this example of an Ag85C protein. In one embodiment, an Ag85C protein includes a substitution mutation of one or more amino acids of all N-glycosylation consensus sequons present in the Ag85C protein.

Other examples of A85 proteins of the present disclosure include those having structural similarity with the amino acid sequence of one of SEQ ID NOs:1-3. As used herein, a protein may be “structurally similar” to a reference protein if the amino acid sequence of the protein possesses a specified amount of structural similarity and/or structural identity compared to the reference protein. Thus, a protein may have structural similarity to a reference protein if, compared to the reference protein, it possesses a sufficient level of amino acid structural identity, amino acid structural similarity, or a combination thereof. Methods for determining whether a protein has structural similarity with the amino acid sequence of one of SEQ ID NOs:1-3 are described herein.

The amino acid sequence of a protein having structural similarity to one of SEQ ID NOs:1-3 can include one or more conservative substitutions of amino acids present in one of SEQ ID NOs:1-3. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a polyprotein. For the purposes of this disclosure, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids.

Whether a protein is structurally similar to a protein of one of SEQ ID NOs:1-3 can be determined by aligning the residues of the two proteins (for example, a candidate protein and any appropriate reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. In one embodiment, a reference protein is a protein described at one of SEQ ID NOs:1-3 but includes the substitution mutation(s) described herein to reduce glycosylation. In one embodiment, a reference protein is a wild type Ag85A, Ag85B, or Ag85C protein. The amino acid sequence of wild type Ag85A, Ag85B, and Ag85C proteins are readily available to the person of ordinary skill. A candidate protein is the protein being compared to the reference protein. A candidate protein can be produced using recombinant techniques, or chemically or enzymatically synthesized.

Unless modified as otherwise described herein, a pair-wise comparison analysis of amino acid sequences can be carried out using the Blastp program of the Blastp suite-2sequences search algorithm, as described by Tatusova et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence:11 extension:1, compositional adjustments=conditional compositional score matrix adjustment. Alternatively, proteins may be compared using other commercially available algorithms, such as the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions.

Thus, as used herein, reference to an amino acid sequence disclosed at one of SEQ ID NOs:1-3 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to an amino acid sequence disclosed at one of SEQ ID NOs: 1-3 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Unless a specific level of sequence similarity and/or identity is expressly indicated herein (e.g., at least 80% sequence similarity, at least 90% sequence identity, etc.), reference to the amino acid sequence of an identified SEQ ID NO includes variants having sequence similarity or sequence identity of at least 80%. An Ag85 protein can include portion of the mature protein. For instance, an Ag85 protein of the present disclosure includes those having a truncation at the amino-terminal end, the carboxy-terminal end, or both. The truncation can be at least 1, at least 2, at least 3, at least 4, at least 5, at least 6, at least 7, at least 8, at least 9, at least 10, at least 11, at least 12, at least 13, at least 14, or at least 15 amino acids from one or both ends of the protein, and no greater than 15, no greater than 14, no greater than 13, no greater than 12, no greater than 11, no greater than 10, no greater than 9, no greater than 8, no greater than 7, no greater than 6, no greater than 5, no greater than 4, no greater than 3, no greater than 2, or no greater than 1 amino acids from one or both ends of the protein. An Ag85 protein of the present disclosure having a truncation has immunogenic activity comparable to an Ag85 protein described herein that has no glycosylation sites.

An Ag85 protein described herein can be expressed as a fusion protein that includes an Ag85 protein described herein and heterologous amino acids. In one embodiment, the additional amino acids may be useful in increasing secretion of an Ag85 protein by a cell, such as a eukaryotic cell, e.g., a signal sequence. An example of a useful signal sequence includes, but is not limited to, an IgE signal sequence. In one embodiment, the additional amino acid sequence may be useful for purification of the fusion protein by affinity chromatography. Amino acid sequences useful for purification can be referred to as a tag, and include but are not limited to a polyhistidine-tag (His-tag) and maltose-binding protein. Representative examples may be found in Hopp et al. (U.S. Pat. No. 4,703,004), Hopp et al. (U.S. Pat. No. 4,782,137), Sgarlato (U.S. Pat. No. 5,935,824), and Sharma Sgarlato (U.S. Pat. No. 5,594,115). Various methods are available for the addition of such affinity purification moieties to proteins. Optionally, the additional amino acid sequence, such as a His-tag, can then be cleaved.

Protein described herein may be produced using recombinant DNA techniques, such as an expression vector present in a cell (e.g., a genetically modified cell described herein). Such methods are routine and known in the art. A protein produced using recombinant techniques may be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity.

### Activity

An Ag85 protein of the present disclosure has immunogenic activity (IA). IA activity includes the ability to elicit a humoral immune response (including production of IgG), and/or the ability to elicit a T cell response (as measured by, for instance, interleukin-2 production).

Whether a protein has IA can be determined by in vitro or in vivo assays. In one embodiment, assays for antibody production and interleukin-2 production can be carried out as described in Example 1. In one embodiment, the antibody production resulting from an Ag85 protein described herein is significantly greater than antibody production resulting from a glycosylated Ag85 protein, for instance one that includes one or more N-glycosylated consensus sequons and was expressed by a eukaryotic cell. Optionally, the antibody production resulting from an Ag85 protein described herein is similar to the antibody production resulting from a wild-type Ag85 protein. In one embodiment, the interleukin-2 production resulting from an Ag85 protein described herein is significantly greater than interleukin-2 production resulting from a glycosylated Ag85 protein, for instance one that includes one or more N-glycosylated consensus sequons and was expressed by a eukaryotic cell. Optionally, the interleukin-2 production resulting from an Ag85 protein described herein is similar to the interleukin-2 production resulting from a wild-type Ag85 protein

### Polynucleotides

The present disclosure also includes isolated polynucleotides encoding a protein described herein. A polynucleotide encoding a protein described herein can include a nucleotide sequence encoding a protein having the amino acid sequence shown in any one of SEQ ID NOs:1-3 with one or more of the substitution mutations described herein, or a protein that is structurally similar. A nucleotide sequence of a polynucleotide encoding a protein described herein can be readily determined by one skilled in the art by reference to the standard genetic code, where different nucleotide triplets (codons) are known to encode a specific amino acid. As is readily apparent to a skilled person, the class of nucleotide sequences that encode any protein described herein is large as a result of the degeneracy of the genetic code, but it is also finite.

The skilled person will recognize that the polynucleotides of the present disclosure encompass an RNA or a DNA sequence encoding a protein described herein, and any modified forms thereof, including chemical modifications of the DNA or RNA which render the nucleotide sequence more stable when it is cell free or when it is associated with a cell. In one embodiment, a polynucleotide is DNA, or RNA, or a combination thereof, containing a coding region encoding a protein described herein. In one embodiment, a polynucleotide is a messenger RNA (mRNA) that includes a coding region encoding a protein described herein.

Polynucleotides of the present invention may be modified. Such modifications can be useful to enhance the efficiency with which a nucleotide sequence is taken up by a cell and/or increase stability of the polynucleotide in certain environments. Modifications can include a nucleic acid sugar, base, or backbone, or any combination thereof. The modifications can be synthetic, naturally occurring, or non-naturally occurring. A polynucleotide of the present invention can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide-nucleic acids. Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides. Other examples of modifications are known to the skilled person (U.S. Published Patent Application 2016/0331828).

A polynucleotide encoding a protein described herein can be present in a vector. A vector is a replicating polynucleotide, such as plasmid, phage, viral, or cosmid, to which another polynucleotide may be attached so as to bring about the replication of the attached polynucleotide. Construction of vectors containing a polynucleotide employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the coding region present on the polynucleotide, i.e., an expression vector. The term vector includes, but is not limited to, plasmid vectors, viral vectors, cosmid vectors, and artificial chromosome vectors. Examples of viral vectors include, for instance, adenoviral vectors, adeno-associated viral vectors, lentiviral vectors, retroviral vectors, and herpes virus vectors. In one embodiment, a vector is capable of replication in a microbial host, for instance, a prokaryotic bacterium, such as E. coli. In one embodiment, a vector is capable of replication in a eukaryotic host, for instance, a cell of a mammalian cell line.

Selection of a vector depends upon a variety of desired characteristics in the resulting construct, such as a selection marker, vector replication rate, and the like. Suitable host cells for cloning or expressing the vectors herein include prokaryotic and eukaryotic cells. Suitable prokaryotic cells include eubacteria, such as gram-negative microbes, for example, E. coli. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

An expression vector optionally includes regulatory sequences operably linked to the coding region. Regulatory sequences include, but are not limited to, promoters, enhancers, transcription initiation sites, translation start sites, translation stop sites, transcription terminators, and poly(A) signals. mRNA molecules typically regulatory sequences operably linked to the coding region including, but not limited to, a 5′ untranslated region (UTR), a 3′ UTR, a 5′ cap, and a poly-A tail. Nucleotide sequences that can be used as 5′ UTR, 3′ UTR, 5′ cap, and poly-A tail are known in the art and can be readily adapted for use in a mRNA encoding a protein disclosed herein.

The disclosure is not limited by the use of any particular regulatory sequence, and a wide variety of regulatory sequences are known. A regulatory sequence may be one naturally associated with a coding region encoding a wild-type Ag85 protein. Such a regulatory sequence is referred to as “endogenous.” Alternatively, a coding region can be positioned to be under the control of a recombinant or heterologous regulatory sequence such as a promoter, e.g., a promoter that is not normally associated with a coding region encoding a wild-type Ag85 protein in its natural environment, i.e., in a M. tuberculosis cell. A heterologous enhancer refers also to an enhancer not normally associated with a coding region encoding a wild-type Ag85 protein in its natural environment. A heterologous regulatory sequence may include regulatory sequences of other coding regions, and regulatory sequences isolated from any other prokaryotic cell, virus, or eukaryotic cell, and regulatory sequences not “naturally occurring,” e.g., containing different elements of different transcriptional regulatory regions, and/or mutations that alter expression. In addition to producing nucleic acid sequences of regulatory sequences synthetically, sequences may be produced using recombinant cloning and/or nucleic acid amplification technology, including PCR, in connection with the compositions disclosed herein (U.S. Pat. Nos. 4,683,202, 5,928,906). Furthermore, the control sequences that direct transcription and/or expression of sequences within non-nuclear organelles such as mitochondria, chloroplasts, and the like, can be employed as well.

The skilled person will recognize that the regulatory sequences used are those that effectively direct the expression of the protein encoded by the DNA or RNA polynucleotide in the cell type, organelle, and/or organism chosen for expression. Those of skill in the art of molecular biology generally know how to use regulatory sequences and cell type combinations for expression of a coding region to result in protein production. The promoters employed may be constitutive, tissue-specific, inducible, and/or useful under the appropriate conditions to direct high level expression of the introduced DNA segment, such as is advantageous in the large-scale production of Ag85 proteins, and in expression of Ag85 proteins in cells of a subject to result in an immune response to the Ag85.

An expression vector may optionally include a ribosome binding site and a start site (e.g., the codon ATG) to initiate translation of the transcribed message to produce the protein. It may also include a termination sequence to end translation. The polynucleotide used to transform the host cell may optionally further include a transcription termination sequence.

Vectors may include constitutive, inducible, and/or tissue specific promoters for expression of a coding region present on a polynucleotide of the present invention in a particular tissue or intracellular environment, examples of which are known to one of ordinary skill in the art. Constitutive mammalian promoters include, but are not limited to, polymerase promoters as well as the promoters for the following genes: hypoxanthine phosphoribosyl transferase (HPTR), adenosine deaminase, pyruvate kinase, and β-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art.

Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, cardiac tissue specific promoters. Another tissue-specific promoter includes the promoter for creatine kinase, which has been used to direct expression in muscle and cardiac tissue and immunoglobulin heavy or light chain promoters for expression in B cells.

Other tissue specific promoters include the human smooth muscle α-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, β-galactosidase α-2,6 sialylkansferase promoter, insulin-like growth factor binding protein (IGFBP-1) promoter, aldolase B promoter, human transferrin promoter, and collagen type I promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter. Exemplary tissue-specific expression elements for breast cells include, but are not limited to, the human alpha-lactalbumin promoter. Exemplary tissue-specific expression elements for the lung include, but are not limited to, the cystic fibrosis transmembrane conductance regulator (CFTR) gene promoter.

A vector introduced into a host cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence are sequences that confer resistance to kanamycin, ampicillin, chloramphenicol, tetracycline, and neomycin.

In some embodiments, a coding region present on a polynucleotide is expressed in a eukaryotic cell. A polynucleotide can undergo codon optimization to improve expression in a given eukaryotic cell. For example, the polynucleotide can be codon-optimized for human expression. In addition to optimizing a polynucleotide for expression in a eukaryotic cell, a polynucleotide can be further optimized to correctly reflect GC content of a host cell, increase stability, reduce secondary structures, minimize tandem repeat codons, and/or minimize base runs that may impair expression.

In another embodiment, the expression vector that includes the polynucleotide can include a Kozak element to initiate translation. In another embodiment, the nucleic acid is removed of cis-acting sequence motifs/RNA secondary structures that would impede translation. Such modifications, and others, are known in the art for use in nucleic acid vaccines (Kutzler et al, 2008, Nat. Rev. Gen. 9: 776-788; International Patent Application No. PCT/US2007/000886; International Patent Application No.; PCT/US2004/018962). U.S. Published Patent Application 2016/0331828.

Polynucleotides can be produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are readily available.

### Genetically Modified Cells

Also provided is a genetically modified cell having a polynucleotide encoding an Ag85 protein described herein. Compared to a control cell that is not genetically modified, a genetically modified cell can exhibit production of an Ag85 protein. A polynucleotide encoding an Ag85 protein may be present in the cell as a vector or integrated into genomic DNA, such as a chromosome or a plasmid, of the genetically modified cell.

A cell can be a eukaryotic cell or a prokaryotic cell. In one embodiment, the cell is a mammalian cell. Examples of eukaryotic cells include a yeast, an insect, and an animal cell. Examples of an animal cell includes a vertebrate cell, such as a mammalian cell. An example of a mammalian cell includes, but is not limited to, HEK293 N-acetylglucosaminyltransferase I (GnTI)-deficient cells. A eukaryotic cell can be in vitro (i.e., in cell culture), ex vivo (i.e., a cell that has been removed from the body of a subject), or in vivo (i.e., within the body of a subject). Ex vivo cells include, for instance, primary cells (e.g., cells that have recently been removed from a subject and are capable of limited growth in tissue culture medium), and cultured cells (e.g., cells that are capable of long term culture in tissue culture medium).

### Compositions

Also provided are compositions that include an active agent. An active agent can be an Ag85 protein described herein, a polynucleotide encoding an Ag85 protein described herein (e.g., an expression vector that includes the polynucleotide, or a DNA or RNA polynucleotide), or a cell expressing or presenting an Ag85 protein described herein. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active agents can also be incorporated into the compositions.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration.

Appropriate unit dosage forms for enteral administration of the compound of the present disclosure include, but are not limited to, tablets, capsules or liquids. Appropriate unit dosage forms for parenteral administration may include intravenous or intraperitoneal administration. Appropriate unit dosage forms for topical administration include, but are not limited to, nasal sprays, metered dose inhalers, dry-powder inhalers or by nebulization.

The active agents of the present disclosure can be provided in unit dosage form wherein each dosage unit contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present disclosure, alone or in combination with other active agents, calculated in an amount sufficient to produce the desired effect, in association with a pharmaceutically acceptable carrier where appropriate. The specifications for the unit dosage forms of the present disclosure depend on the particular effect to be achieved and the particular pharmacodynamics associated with the pharmaceutical composition in the particular host.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide.

Compositions can include sterile aqueous solutions (where water soluble) or dispersions and sterile powders for the extemporaneous preparation of sterile solutions or dispersions. For parenteral administration, suitable carriers include physiological saline, bacteriostatic water, phosphate buffered saline (PBS), and the like. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active agent in the required amount in an appropriate solvent with one or a combination of ingredients routinely used in pharmaceutical compositions, as required, followed by filtered sterilization. Generally, dispersions are prepared by incorporating the active agent into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

A composition including a pharmaceutically acceptable carrier can also include an immunostimulatory agent or a polynucleotide encoding such an agent. Immunostimulatory agents include but are not limited to an additional antigen, an immunomodulator, an adjuvant, or a combination thereof.

An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest. Adjuvants may include, for example, cholera toxin, salmonella toxin, IL-1, IL-2, emulsifiers, muramyl dipeptides, dimethyldiocradecylammonium bromide (DDA), avridine, aluminum hydroxide, oils, saponins, alpha-tocopherol, polysaccharides, emulsified paraffins (available from under the tradename EMULSIGEN from MVP Laboratories, Ralston, Nebr.), ISA-70, RIBI and other substances known in the art.

Oral compositions generally include an inert diluent or an edible carrier. For the purpose of oral therapeutic administration, the active agent can be incorporated with excipients and used in the form of tablets, troches, or capsules, e.g., gelatin capsules. Oral compositions can also be prepared using a fluid carrier. Pharmaceutically compatible binding agents and/or other useful materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation (e.g., topical administration), the active agents can be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

In those embodiments where a polynucleotide encoding the Ag85 protein is administered, any method suitable for administration of a polynucleotide, e.g., an expression vector, DNA polynucleotide, or RNA polynucleotide, can be used. In addition to the routes of administration described herein, e.g., systemic or local, parenteral (e.g., intravenous, intradermal, subcutaneous, intraperitoneal, intramuscular), enteral (e.g., oral), and topical (e.g., epicutaneous, inhalational, transmucosal), methods using physical means, such as electroporation (electricity), a gene gun (physical force) or applying large volumes of a liquid (pressure), and methods wherein polynucleotide is complexed to another entity, such as a liposome, aggregated protein or transporter molecule, can also be used.

The active agents may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art. The active agents may be prepared with carriers that will aid in maintaining the stability of the active agent. Examples of carriers that can aid in maintaining the stability of mRNA polynucleotides are known in the art (U.S. Published Patent Application 2016/0331828).

Toxicity and therapeutic efficacy of the active agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Recombinant proteins exhibiting high therapeutic indices are preferred.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the unit dosage form employed and the route of administration used. For a compound used in the methods described herein, the therapeutically effective dose can be estimated initially from animal models. A dose may be formulated in animal models to achieve a circulating plasma concentration range that includes the IC50 (i.e., the concentration of the test compound which achieves a half-maximal inhibition of signs of disease, such as obesity). Such information can be used to more accurately determine useful doses in humans. Levels in plasma may be measured using routine methods.

The compositions can be administered one or more times per day to one or more times per week, including once every other day. The skilled artisan will appreciate that certain factors may influence the dosage and timing required to effectively treat a subject, including but not limited to the severity of the condition, previous treatments, the general health and/or age of the subject, and other diseases present. Moreover, treatment of a subject with an effective amount of an active agent can include a single treatment or, preferably, can include a series of treatments.

### Methods

The present disclosure provides methods. In one embodiment, a method is for making an Ag85 protein described herein. In one embodiment, the method includes incubating a cell under suitable conditions for expression of an Ag85 protein. The cell is a genetically modified cell, and in one embodiment is a genetically modified eukaryotic cell. Examples of eukaryotic cells include a yeast, an insect, and an animal cell. Examples of an animal cell include a vertebrate cell, such as a mammalian cell. Optionally, the method includes introducing into a host cell a vector that includes a coding region encoding an Ag85 protein described herein. In one embodiment, the method includes isolating or purifying an Ag85 protein from a cell or from a medium. In those embodiments where an Ag85 protein includes additional amino acids useful for isolating or purifying the protein, the method can also include cleavage of the additional amino acids from the Ag85 protein.

In one embodiment, a method includes administering to a subject an effective amount of a composition including a protein or polynucleotide described herein. As used herein, an “effective amount” of a composition including a protein or polynucleotide described herein is the amount able to elicit the desired response in the recipient. The subject can be, for instance, murine (e.g., a mouse or rat), or a primate, such as a human.

In one embodiment, a method includes inducing an immune response to the protein or polynucleotide described herein by administering to a subject an effective amount of a composition described herein. In this embodiment, an “effective amount” is an amount effective to result in the production of an immune response in the animal. The immune response can be humoral, cell-based, or a combination thereof. A humoral immune response includes the production of antibodies that are antigen-specific and bind the Ag85 protein used to induce the immune response. Methods for determining whether a subject has produced antibodies that specifically bind an Ag85 protein described herein can be determined using routine methods. A cell-based response includes the production of T cells that produce interleukin-2 after stimulation by the Ag85 protein used to induce the immune response.

As used herein, an antibody that can “specifically bind” a protein is an antibody that interacts with the epitope of the Ag85 that induced the synthesis of the antibody or interacts with a structurally related epitope.

In one embodiment, a method includes treating an infection in a subject caused by Mycobacterium tuberculosis. The subject used in a method described herein can be an animal such as, but not limited to, a human, a murine animal (e.g., a mouse or rat), a Guinea pig, a rabbit, a rhesus monkey, or a macaque. The method includes administering an effective amount of the composition to an animal having an infection caused by M. tuberculosis. Optionally, the method can include determining whether the M. tuberculosis causing the infection has decreased. Methods for determining whether an infection is caused by a M. tuberculosis are routine and known in the art. The infection can be localized or systemic. An example of a localized M. tuberculosis infection is presence in the lungs, kidneys, spine, or brain. In one embodiment, parenteral administration of a composition can be used to reduce a M. tuberculosis infection in a subject. In this aspect of the disclosure, an “effective amount” of a composition described herein is the amount able to elicit the desired response in the recipient, e.g., a reduction in the amount of M. tuberculosis present in a subject. The reduction can be a decrease of at least 2-fold, at least 3-fold, or at least 4-fold in the subject compared to the subject before administering the composition.

In another embodiment, a method includes treating one or more symptoms of certain conditions in an animal that may be caused by a M. tuberculosis infection. M. tuberculosis infection can be Latent TB Infection (LTBI) and TB Disease. A symptom of LTBI is a positive TB skin test reaction or positive TB blood test. Symptoms of TB Disease include coughing that lasts three or more weeks, coughing up blood, chest pain or pain with breathing or coughing, unintentional weight loss, fatigue, fever, and/or night sweats. LTBI does not typically cause any conditions. Examples of conditions caused by TB Disease include tuberculosis of the lungs, including Ghon focus/Ghon's complex, Pott disease; tuberculosis outside of the lungs, including brain, e.g., Meningitis and Rich focus, lymph node, e.g., Tuberculous lymphadenitis and Tuberculous cervical lymphadenitis; cutaneous tuberculosis, e.g., Scrofuloderma, Erythema induratum, Lupus vulgaris, Prosector's wart, Tuberculosis cutis orificialis, Tuberculous cellulitis, and Tuberculous gumma; and others including Lichen scrofulosorum, Tuberculid (Papulonecrotic tuberculid), Primary inoculation tuberculosis, Miliary, Tuberculous pericarditis, Urogenital tuberculosis, Multi-drug-resistant tuberculosis, and Extensively drug-resistant tuberculosis.

Treatment of one or more of these conditions can be prophylactic or, alternatively, can be initiated after the development of a condition described herein. Treatment that is prophylactic, for instance, initiated before a subject manifests a symptom of a condition caused by M. tuberculosis, is referred to herein as treatment of a subject that is “at risk” of developing the condition. Typically, a subject “at risk” of developing a condition is a subject likely to be exposed to a M. tuberculosis. Accordingly, administration of a composition can be performed before, during, or after the occurrence of the conditions described herein. Treatment initiated after the development of a condition may result in decreasing the severity of the symptoms of one of the conditions, including completely removing the symptoms. In this aspect of the disclosure, an “effective amount” is an amount effective to prevent the manifestation of symptoms of a condition, decrease the severity of the symptoms of a condition, and/or completely remove the symptoms.

The potency of a composition described herein can be tested according to standard methods. For instance, the use of mice as an experimental model for M. tuberculosis infection in humans is well established.

### Kits

The present disclosure also provides a kit for administering a composition described herein. In one embodiment, the kit includes a vector that includes a coding region encoding an Ag85 protein in an amount sufficient for administration to a subject in need thereof.

In another embodiment, the present disclosure also provides a kit directed to using an Ag85 protein described herein. In one embodiment, the kit includes an Ag85 protein, isolated or optionally purified, in a suitable packaging material.

In another embodiment, the present disclosure also provides a kit directed to using a polynucleotide encoding an Ag85 protein described herein. In one embodiment, the kit includes a DNA polynucleotide encoding Ag85 protein in a suitable packaging material. In one embodiment, the kit includes a RNA polynucleotide, such as a mRNA polynucleotide, encoding Ag85 protein in a suitable packaging material.

Optionally, other reagents such as buffers or a pharmaceutically acceptable carrier (either prepared or present in its constituent components, where one or more of the components may be premixed or all of the components may be separate), and the like, are also included. In one embodiment, the protein or vector may be present with a buffer, or may be present in separate containers. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label, which indicates that the contents can be used for administration of a vector or an An85 protein. In addition, the packaging material contains instructions indicating how the materials within the kit are used. As used herein, the term “package” refers to a solid matrix or material such as glass, plastic, paper, foil, and the like, capable of holding within fixed limits a vector or a genetically modified cell. Thus, for example, a package can include a glass or plastic vial used to contain appropriate quantities of a vector or protein. “Instructions for use” typically include a tangible expression describing the reagent concentration or at least one method parameter.

### EXEMPLARY EMBODIMENTS

Embodiment 1. An isolated polynucleotide comprising: (a) a nucleotide sequence encoding a protein comprising an amino acid sequence encoding a mature Mycobacterium tuberculosis protein selected from Ag85A, Ag85B, and Ag85C, wherein the protein does not comprise a N-glycosylation consensus sequon, or (b) the full complement of the nucleotide sequence of (a).

Embodiment 2. The isolated polynucleotide of Embodiment 1, wherein the protein encoded by the polynucleotide and the amino acid sequence of SEQ ID NO:1 have at least 80% identity, wherein at least one amino acid of the N-linked glycosylation site NNT at positions 202-204 comprises at least one substitution mutation.

Embodiment 3. The isolated polynucleotide of any one of Embodiments 1-2 wherein the amino acid at position 202 is substituted.

Embodiment 4. The isolated polynucleotide of any one of Embodiments 1-3 wherein the amino acid at position 202 is substituted with Q.

Embodiment 5. The isolated polynucleotide of Embodiment 1, wherein the protein encoded by the polynucleotide and the amino acid sequence of SEQ ID NO:2 have at least 80% identity, wherein at least one amino acid of each of the N-linked glycosylation sites NNS at positions 31-33, NNT at positions 203-205, NGT at positions 213-215, and NGT at positions 259-261 comprises at least one substitution mutation.

Embodiment 6. The isolated polynucleotide of Embodiment 1 or 5 wherein the amino acids at positions 31, 203, 213, and 259 are substituted.

Embodiment 7. The isolated polynucleotide of Embodiment 1, wherein the protein encoded by the polynucleotide and the amino acid sequence of SEQ ID NO:3 have at least 80% identity, wherein at least one amino acid of each of the N-linked glycosylation sites NYT at positions 90-92, NDS at positions 167-169, NNT at positions 201-203, NGT at positions 211-213, NQT at positions 235-237, and NGT at positions 257-259 comprises at least one substitution mutation.

Embodiment 8. The isolated polynucleotide of Embodiment 1 or 7 wherein the amino acids at positions 167, 201, 211, 235, and 257 are substituted.

Embodiment 9. The isolated polynucleotide of any one of Embodiments 1-8, wherein the polynucleotide comprises DNA.

Embodiment 10. The isolated polynucleotide of any one of Embodiments 1-9, wherein the polynucleotide comprises RNA.

Embodiment 11. The isolated polynucleotide of Embodiment 10, wherein the RNA is a mRNA.

Embodiment 12. The isolated polynucleotide of any one of Embodiments 1-11, wherein the polynucleotide further comprises an operably linked heterologous regulatory sequence.

Embodiment 13. A vector comprising the isolated polynucleotide of any one of Embodiments 1-12.

Embodiment 14. A vector comprising encoding the isolated polynucleotide of any one of Embodiments 1-13, wherein the isolated polynucleotide is mRNA.

Embodiment 15. An isolated protein having immunogenic activity, wherein the protein comprises an amino acid sequence, wherein the amino acid sequence and the amino acid sequence of SEQ ID NO:1, 2, or 3 have at least 80% identity, wherein the protein does not comprise a N-glycosylation consensus sequon.

Embodiment 16. An isolated protein having immunogenic activity, wherein the protein comprises an amino acid sequence, wherein the amino acid sequence and the amino acid sequence of SEQ ID NO:1 have at least 80% identity, wherein at least one amino acid of the N-linked glycosylation site NNT at positions 202-204 of SEQ ID NO:1 comprises at least one substitution mutation, and wherein the protein is not glycosylated.

Embodiment 17. The protein of Embodiments 15 or 16 wherein the amino acid at position 202 is substituted.

Embodiment 18. The protein of any one of Embodiments 15-17 wherein the amino acid at position 202 is substituted with Q.

Embodiment 19. An isolated protein having immunogenic activity, wherein the protein comprises an amino acid sequence, wherein the amino acid sequence and the amino acid sequence of SEQ ID NO:2 have at least 80% identity, wherein at least one amino acid of each of the N-linked glycosylation sites NNS at positions 31-33, NNT at positions 203-205, NGT at positions 213-215, and NGT at positions 259-261 of SEQ ID NO:2 comprises at least one substitution mutation, and wherein the protein is not glycosylated.

Embodiment 20. The protein of claim Embodiments 15 or 19 wherein the amino acids at positions 31, 203, 213, and 259 are substituted.

Embodiment 21. An isolated protein having immunogenic activity, wherein the protein comprises an amino acid sequence, wherein the amino acid sequence and the amino acid sequence of SEQ ID NO:3 have at least 80% identity, wherein at least one amino acid of each of the N-linked glycosylation sites NYT at positions 90-92, NDS at positions 167-169, NNT at positions 201-203, NGT at positions 211-213, NQT at positions 235-237, and NGT at positions 257-259 of SEQ ID NO:3 comprises at least one substitution mutation, and wherein the protein is not glycosylated.

Embodiment 22. The protein of Embodiment 15 or 21 wherein the amino acids at positions 167, 201, 211, 235, and 257 are substituted.

Embodiment 23. A genetically modified eukaryotic cell comprising an exogenous polynucleotide, wherein the exogenous polynucleotide is the polynucleotide of any one of Embodiments 1-14.

Embodiment 24. A genetically modified eukaryotic cell comprising an exogenous polynucleotide encoding the protein of any one of Embodiments 15-22.

Embodiment 25. The genetically modified cell of Embodiment 23 or 24 wherein the cell is a mammalian cell.

Embodiment 26. A composition comprising the polynucleotide of any one of Embodiments 1-14 or the protein of any one of Embodiments 15-22.

Embodiment 27. The composition of claim 26 further comprising a pharmaceutically acceptable carrier.

Embodiment 28. The composition of Embodiment 26 or 27 further comprising an adjuvant.

Embodiment 29. A method for inducing an immune response comprising:
administering to a subject an amount of the composition of any one of Embodiments 26-28 effective to induce the subject to produce an immune response to the protein encoded by the polynucleotide.

Embodiment 30. The method of Embodiment 29 wherein the immune response comprises a humoral immune response.

Embodiment 31. The method of Embodiments 29 or 30 wherein the immune response comprises a cell-mediated immune response.

Embodiment 32. A method for treating an infection in a subject, the method comprising:
administering an effective amount of the composition of claim 26 to a subject having or at risk of having an infection caused by Mycobacterium tuberculosis.

Embodiment 33. The method of Embodiment 32 wherein the subject is a mammal.

Embodiment 34. The method of Embodiments 32 or 33 wherein the mammal is a human.

Embodiment 35. The isolated polynucleotide of any one of Embodiments 1-14 for use in therapy.

Embodiment 36. The isolated polynucleotide of any one of Embodiments 1-14 for use as a medicament.

Embodiment 37. The isolated polynucleotide of any one of Embodiments 1-14 for use in the treatment of tuberculosis.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Host protein glycosylation in nucleic acid vaccines as a major hurdle in vaccine design

Nucleic acid vaccines introduce the genetic materials encoding antigenic proteins into host cells. These proteins of microbial origin will be subject to modification by host's glycosylation machinery if their amino acid sequences contain glycosylation sites. The presence of host glycans on the generated protein may prevent a strong protective immune response either through hindering access to key epitopes by lymphocytes or through altering immune responses by binding to immunoregulatory glycan binding receptors on immune cells. Ag85A expressed by Mycobacterium tuberculosis (Mtb) is a highly immunogenic bacterial surface protein that is commonly used in nucleic acid vaccines in multiple clinical trials. Here we show that when Ag85A is expressed in mammalian cells, it is glycosylated, does not induce a strong humoral immune response in mice, and does not activate Ag85A specific lymphocytes as highly as Ag85A natively expressed by the bacterium. Our study indicates that host glycosylation of the vaccine target can impede its antigenicity and immunogenicity. Glycosylation of the antigenic protein targets therefore must be carefully evaluated in designing nucleic acid vaccines.

### Introduction

DNA vaccines, recombinant vector vaccines and mRNA vaccines comprise nucleic acid vaccines (1). This next-generation vaccine approach adopts a different modus operandi than other vaccine types in use today: rather than using whole pathogens or purified protein products, these vaccines introduce the genetic materials encoding antigenic, pathogen-associated proteins into the host cells. Unlike viral proteins, proteins of non-viral pathogens are not expressed in host cells upon infection. We postulated that when nucleic acid vaccines are used for non-viral pathogens, the proteins expressed by host cells would be structurally and immunogenically different from native pathogen protein due to the elaboration of host post-translational modifications (PTMs), especially glycosylation for targets that enter the secretory pathway. Hence, an immune response would not be raised against these recombinant proteins expressed by host cells to the same degree as native proteins, leading to poorly immunogenic vaccines. Furthermore, glycans on glycoproteins are recognized by many regulatory immune cell receptors (2). Therefore, the glycans on the proteins expressed through nucleic acid vaccines may influence the immune responses against the antigen. Here, we investigated the impact of host glycosylation on the immunological properties of Mtb surface protein, Ag85A, a target protein used in multiple clinical vaccine trials (3). In two recent clinical trials, a viral vector-based Ag85A vaccine candidate was not protective among the participants (4, 5). Similarly, another viral vector-based vaccine candidate carrying the gene of Ag85A failed to generate high immunogenicity in another clinical trial (6). Toward understanding the failure of these strategies, we show that Ag85A expressed in mammalian cells is glycosylated and immunization with this recombinant Ag85A generates a significantly reduced immune response compared to immunizations with its native form expressed in Mtb.

### Materials and Methods

### Ethics Statement

All mouse experiments were in compliance with the University of Georgia Institutional Animal Care and Use Committee under an approved animal use protocol. Our animal use protocol adheres to the principles outlined in U.S. Government Principles for the Utilization and Care of Vertebrate Animals Used in Testing, Research and Training, the Animal Welfare Act, the Guide for the Care and Use of Laboratory Animals, and the AVMA Guidelines for the Euthanasia of Animals.

### Proteins and Bacteria

Native Ag85A purified form Mtb, strain H37Rv and gamma irradiated Mtb, strain H37Rv, were obtained from BEI resources (NIH Biodefense and Emerging Infections Research Resources Repository, National Institute of Allergy and Infectious Diseases, NIH).

### Expression of Recombinant Ag85As

To generate the plasmid for the expression of Ag85A in mammalian cells, in a polymerase chain reaction (PCR) tissue plasminogen activator leader sequence was placed in front of Ag85A gene without its signal sequence and the end product was inserted into pGEc2-DEST vector.

To generate the plasmid for N246Q Ag85A, the codon of asparagine-246 was mutated to a codon of glutamine by QuikChange II Site-Directed Mutagenesis Kit, Agilent and the gene of GFP is placed at the 3′ end with a TEV protease cleavage site between the two.

For the expression of wild type and N246Q Ag85A in mammalian cells, human embryonic kidney cells (HEK 293-F) were cultured in FreeStyle 293 expression medium. Cells were transiently transfected with Polyethylenimine (MW 25,000). 293-F Ag85A (wild type) and N246Q Ag85A-GFP were purified using Ni-NTA affinity columns. N246Q Ag85A was cleaved from the fusion protein by TEV protease and purified from GFP and the enzyme.

### Generation of Adenovirus 5 as a Vector for Ag85A

To generate adenoviruses for the expression of Ag85A we utilized AdEasy system (Luo et al. 2007, Nat Protoc 2(5):1236-1247). First, the gene of Ag85A with tissue plasminogen activator leader sequence was inserted into pAdTrack-CMV vector. Then, the vector was linearized and electroporated into AdEasier cells where the gene of Ag85A was transferred into pAdEasy-1. After selecting AdEasier cells with desired homologous recombination on kanamycin plates, pAdEasy-1 plasmids were purified, linearized and transfected into HEK 293T cells. After two weeks from transfection day viruses were harvested and used to infect HEK 293 cells.

### Protein Deglycosylation

293-F Ag85A was denatured at 95° C. for 5 min in a denaturing solution with 0.05% SDS and 0.05 M 2-mercaptoethanol. The solution was cooled down to room temperature and added detergent, NP-40 with a 0.15 final percentage. After PNGase F was added, the reaction mixture was incubated at 37° C. for 16 hr.

### Western Blot and Lectin Blots

Proteins were run in a Bio-RAD 4-20% Mini-PROTEAN® TGX Stain-Free™ protein gel and visualized by a Bio-RAD Gel Doc EZ System imager. Subsequently, proteins on the gel were transferred to PVDF membrane and probed with serum from mice immunized with 293-F Ag85A. Immunoreactive bands were visualized by SuperSignal West Pico Plus (ThermoFisher Scientific) and the image was captured by Analytic Jena imager.

For the lectin blot experiments, biotinylated concanavalin A (Con A, Vector labs) or Sambucus nigra lectin (SNA, Vector labs) was used to probe for glycans on the proteins.

### Monosaccharide Analysis

Proteins were incubated at 100° C. in 2 N Trifluoroacetic acid (TFA) for four hours to hydrolyze and release any glycans. Water and TFA in the solution were evaporated in a speedvac. Monosaccharides were dissolved in water and analyzed in a high pH anion-exchange chromatography with pulsed amperometric detection (HPAEC-PAD).

### Mice and Immunizations

Eight-week-old female BALB/c mice were obtained from Taconic Biosciences. Groups of mice were immunized twice with 10 ug (micrograms) of protein/dose in 250 ug dimethyldioctadecylammonium bromide (DDA, Avanti®)/dose. Immunizations were performed at 2-week intervals subcutaneously along the dorsal plane. Sera was collected one week after booster immunization.

### Enzyme-Linked Immunosorbent Assay (ELISA)

To test Ag85A specific antibodies in mice sera, ELISA plates were coated with lug Ag85A/ml in carbonate buffer at 4° C. overnight. After blocking unoccupied sites with 1% BSA, serum dilutions were applied to the wells for 2 hours at room temperature. Goat anti-mouse IgG-HRP (BioLegend, Cat#405301) was used as a secondary antibody.

To quantify serum antibody titers against Mtb, firstly, ELISA plates were coated with gamma irradiated whole bacteria suspension in PBS. Unoccupied sites were blocked with 1% BSA, and serum dilutions were applied to the wells. Then, secondary antibody was applied.

To quantify IL-2 secretions in in vitro stimulation experiments, plates were coated with anti-IL-2 capture antibodies (BioLegend, Cat# 503704) in carbonate buffer. Media from stimulated cell culture was applied to wells along with cytokine standards. Then, biotinylated detection antibodies (BioLegend, Cat# 503804) and avidin-HRP (BioLegend, Cat#405103) were added to the wells.

### Flow Cytometry Analysis for Mtb Reactive Serum Antibodies

Gamma irradiated bacteria was pelleted and resuspended in FACS buffer (0.1% BSA). 1:100 of pooled sera of the same group was added to the cell suspension. After 30 minutes incubation at 4° C. cell suspension was washed twice with cold FACS buffer. Cells were resuspended in 100 ul (microliter) FACS buffer. Alexa Fluor 647 goat anti-mouse IgG antibodies (BioLegend, Cat# 405322) was added to each tube and incubated at 4° C. for 30 minutes. After cells were washed, they were analyzed with Beckman Coulter Cytoflex flow cytometer in FACS buffer.

### Opsonophagocytosis Assay (OPA)

J774 mouse macrophage cells (8×10⁵ cells/well, 24 well plate) were seeded 16 hours prior to coincubation with bacteria. 10¹⁰ Mtb cells (Strain H37Ra) were incubated in 500 ul of FITC dye with 1 mg/ml concentration in 0.1 M carbonate buffer for 2 hours at room temperature. After being washed, Mtb cells were separated into three tubes and incubated with 500 times diluted mice sera from three immunizations for an hour.

### In vitro Stimulation of Splenocytes

Five weeks following booster immunization, mice were sacrificed, and spleens were collected. Spleens were digested manually and filtered through 40 um (micrometer) cell strainers to obtain a single cell suspension. Red blood cells were lysed using ACK lysis buffer. 2×10⁶ cells/well were seeded in duplicates in a 24 well plate with 10 ug antigen/ml as a stimulant. After 3 days culture media was collected and used in ELISA to quantify IL-2 secreted by splenocytes.

### N-Glycan Analysis

293-F Ag85 was subjected to SDS-PAGE and the resulting gel was stained with Coomassie to visualize the protein. The band corresponding to Ag85 was excised, destained, and treated with DTT and iodoacetamide. The gel piece was cut into smaller cubes and N-glycans were released by in-gel PNGaseF treatment as previously described (Kuster et al. 1997, Anal Biochem 250(1):82-101). Released glycans were extracted from the gel pieces and permethylated prior to analysis by mass spectrometry using direct infusion into a nanospray ionization linear and orbital ion trap instrument (NSI-MSn, LTQ/OrbiTrap ThermoFisher Discovery) (Aoki K, et al. 2007, J Biol Chem 282(12):9127-9142; Anumula and Taylor, 1992, Anal Biochem 203(1):101-108). Graphical representations of monosaccharide residues are consistent with the symbol nomenclature for glycans (SNFG), which has been broadly adopted by the glycomics community (Varki et al. 2015, Glycobiology 25(12):1323-1324).

### Results

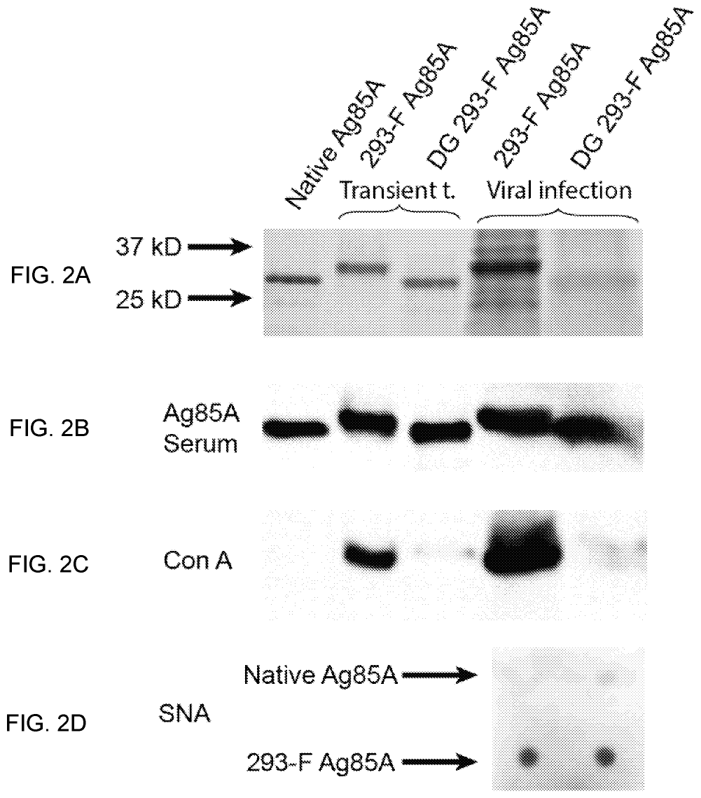

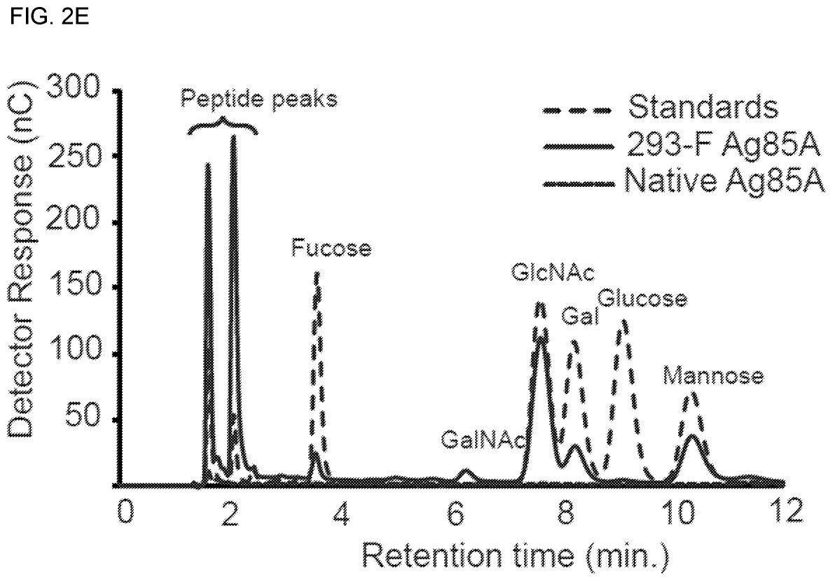

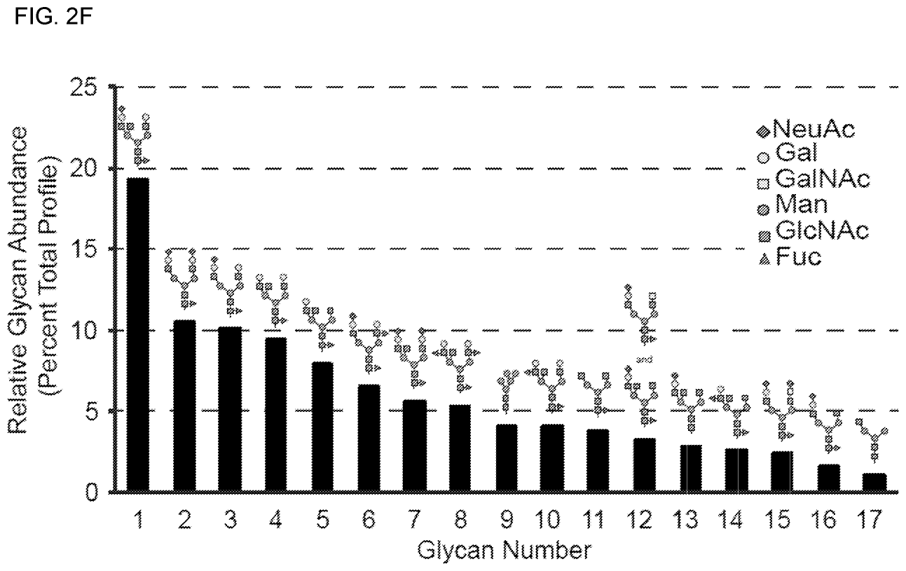

To investigate the effects of host glycosylation on immune responses, we expressed Ag85A in human embryonic kidney cells (HEK 293-F) and designated it 293-F Ag85A. We expressed 293-F Ag85A in two ways. First, HEK 293-F cells were infected with a replication-defective adenovirus 5 expressing Ag85A. Second, we used a transient transfection of HEK 293-F cells. Since the amino acid sequence of Ag85A includes an N-glycosylation consensus sequon and the molecular weights of the 293-F Ag85As expressed through transient transfection or viral vector infection are higher than native Ag85A (FIG. 2A), we expected the recombinant protein to be N-glycosylated. To confirm the presence of an N-glycan, both 293-F Ag85As were treated with the enzyme, PNGase F, which removes N-glycans on proteins expressed in vertebrate cells. 293-F Ag85As treated with PNGase F showed a molecular weight shift (FIG. 2A) indicating that 293-F Ag85A is N-glycosylated. To confirm that the major band displayed in FIG. 2A for Ag85A expressed through viral infection is in fact Ag85A, western blotting was performed probing with serum from mice immunized with purified 293-F Ag85A (FIG. 2B). In a lectin blotting experiment concanavalin A (ConA), a lectin that binds to mannose (and with less avidity to glucose) residues on N-linked glycans reacted with 293-F Ag85A but not native Ag85A (FIG. 2C). However, upon deglycosylation by PNGase F, 293-F Ag85A binding is ablated (FIG. 2C). As it is often indicated in immunoregulation (2, 7, 8), we next confirmed the presence of terminal sialic acid on 293-F Ag85A in a dot blot by probing with SNA, a lectin that binds to a2,6-linked sialic acids (FIG. 2D). Monosaccharide composition of glycans of native and 293-F Ag85A were then analyzed by high performance anion exchange chromatography (HPAEC) with pulsed amperometric detection (PAD) after acid hydrolysis, which revealed the presence of GalNAc, GlcNAc, mannose, galactose, and fucose as the neutral monosaccharides on the 293-F Ag85A, while no monosaccharide is detected on native Ag85A (FIG. 2E). To acquire a higher-resolution understanding of 293-F Ag85A glycosylation, its N-glycans were released by PNGaseF digestion, permethylated to enhance structural characterization, and analyzed by multi-dimensional mass spectrometry (NSI-MSn). Among the detected glycans, 15 of 17 were complex type, modified with core and external fucose and variably terminated with sialic acid (NeuAc) (FIG. 2F).

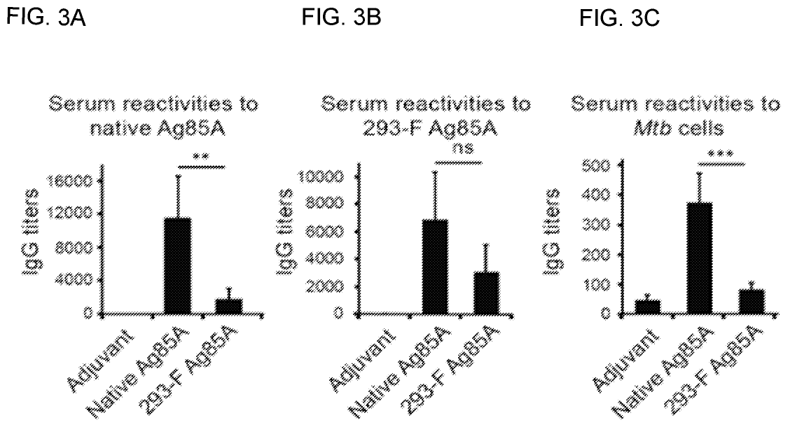

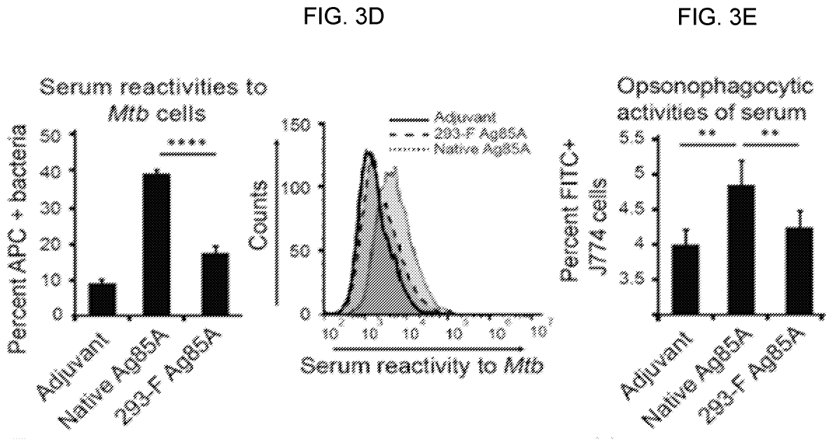

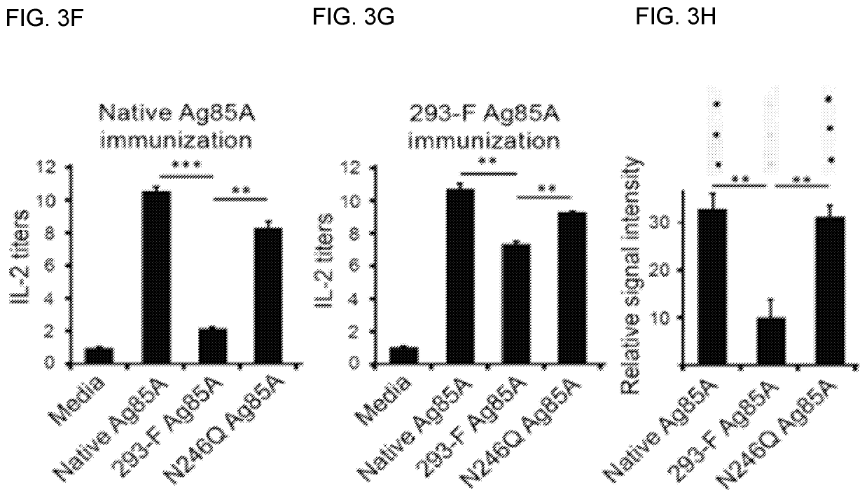

To investigate the differences in antigenicity and immunogenicity of native and 293-F Ag85A, three groups of mice were immunized with adjuvant alone (250 μg dimethyldioctadecylammonium bromide (DDA, Avanti®)/dose), native Ag85A, or 293-F Ag85A (10 μg of protein/dose). A week after the booster immunization, sera were collected from immunized mice and antibody reactivities against native or 293-F Ag85A were tested by ELISA. Immunization of mice with native Ag85A yielded significantly higher IgG titers than sera from 293-F Ag85A immunized mice when plates are coated with native Ag85A (FIG. 3A) or 293-F Ag85A (FIG. 3B). We next tested the serum IgGs from immunized mice for their binding to bacterial cells in a whole cell ELISA. Sera obtained from native Ag85A-immunized mice showed significantly higher IgG binding to plates coated with gamma irradiated Mtb than sera from 293-F Ag85A immunized mice (FIG. 3C). The differential binding of serum antibodies to bacteria observed in ELISA was also confirmed by flow cytometry (FIG. 3D). These results demonstrate that immunization with 293-F Ag85A does not evoke a humoral immune response to bacteria as strongly as immunization with native Ag85A does. One of the effector functions of antigen-specific antibodies is to enhance phagocytosis and clearance of pathogens. Therefore, we compared the function of antibodies raised against native or 293-F Ag85A in an opsonophagocytosis assay (OPA). For this assay, we incubated FITC-labeled Mtb with sera from different immunization groups. Opsonized bacteria were then incubated with J774 mouse macrophages. Flow cytometry was used to quantify FITC⁺ J774 cells, indicative of phagocytosed bacteria. In parallel to the sera reactivities against whole bacteria, serum from native Ag85A immunization induced a significantly higher degree of binding/uptake of bacteria compared to serum from 293-F Ag85A immunization (FIG. 3E). To test Ag85A-specific interleukin-2 (IL-2) production as a measure of lymphocyte activation, splenic mononuclear cells harvested from mice immunized with native Ag85A or 293-F Ag85A were cultured in the presence of native or 293-F Ag85A (10 μg antigen/ml) and after 3 days the IL-2 levels in the culture media were quantified. Native Ag85A stimulation induced significantly higher IL-2 secretion than 293-F Ag85A stimulation regardless of which Ag85A variant was used in the immunization (FIG. 3F and 3G). To assess the recovery of T cell stimulation by the removal of N-linked glycan, we expressed Ag85A in HEK 293-F cells in its non-glycosylated form after mutagenesis of the N-glycosylation site (N246Q Ag85A). As demonstrated in FIGS. 2F and 2G, N246Q Ag85A induces IL-2 responses. We finally tested serum IgGs from native Ag85A-immunized mice for their binding to native, 293-F or N246Q Ag85A and showed that native Ag85A serum IgGs react with 293-F Ag85A significantly less than either native or N246Q Ag85A (FIG. 3H).

### Discussion

Since their introduction, nucleic acid vaccines have shown great promise and have been quickly propelled to clinical trials (9). The utilization of host's protein biosynthesis machinery through introduction of the antigenic gene potentially provides variety of advantages over traditional subunit vaccines including vaccine cost-effectiveness, stability and persistence of immunogenicity. However, relying on host's cellular machinery to produce the antigens comes with a major potential caveat: mammalian post-translational machinery—namely glycosylation—may decorate the antigenic protein with self glycans. This may in turn yield undesirable antigens for immune recognition and poor immunogens for eliciting protective immune responses. Moreover, decoration of antigenic protein with host glycans may trigger suppressive immune responses since host glycans are known for their immunoregulatory properties (2, 7, 8). The downregulatory effects of host-associated glycans are also exploited by many pathogens, which display these structures on their surfaces to mimic “self” and thus evade immune recognition (10-14).

Through the use of a clinical nucleic acid vaccine candidate, Ag85A, this study demonstrates that when nucleic acid vaccines are used to express a bacterial protein in mammalian host cells, the protein product can be glycosylated by host glycosylation machinery. In turn, these glycoprotein antigens may elicit impaired and/or tolerogenic immune responses due to their dampened antigenicity and immunogenicity or their newly acquired immunosuppressive properties. Thus, glycosylation is a critical posttranslational modification to consider in designing vaccine strategies. An ideal vaccine target has to share the same antigenic determinants as the native antigen expressed on the surface of the pathogen. Removal of N-linked sequons is potentially a reasonable strategy toward avoiding this complication. Moving vaccine candidates from preclinical investigation to clinical phase studies comes with substantial cost and effort. We believe the findings of this study serve as essential criteria for the design of future nucleic acid vaccines and projecting their ultimate success of protectivity.

### Citations for Example 1

1. W. R. Ellis, R. Rappuoli, S. Ahmed, “Technologies for making new vaccines” in Vaccines, S. A. Plotkin, W. A. Orenstein, P. A. Offit, Eds. (Saunders, 2013), chap. 60, pp. 1182-1199.

2. J. Y. Zhou, D. M. Oswald, K. D. Oliva, L. S. C. Kreisman, B. A. Cobb, The Glycoscience of Immunity. Trends Immunol 39, 523-535 (2018).

3. G. Voss et al., Progress and challenges in TB vaccine development. F1000Res 7, 199 (2018).

4. M. D. Tameris et al., Safety and efficacy of MVA85A, a new tuberculosis vaccine, in infants previously vaccinated with BCG: a randomised, placebo-controlled phase 2b trial. Lancet 381, 1021-1028 (2013).

5. B. P. Ndiaye et al., Safety, immunogenicity, and efficacy of the candidate tuberculosis vaccine MVA85A in healthy adults infected with HIV-1: a randomised, placebo-controlled, phase 2 trial. Lancet Respir Med 3, 190-200 (2015).

6. M. Tameris et al., A double-blind, randomised, placebo-controlled, dose-finding trial of the novel tuberculosis vaccine AERAS-402, an adenovirus-vectored fusion protein, in healthy, BCG-vaccinated infants. Vaccine 33, 2944-2954 (2015).

7. A. Varki, Since there are PAMPs and DAMPs, there must be SAMPs? Glycan “self-associated molecular patterns” dampen innate immunity, but pathogens can mimic them. Glycobiology 21, 1121-1124 (2011).

8. M. S. Macauley et al., Antigenic liposomes displaying CD22 ligands induce antigen-specific B cell apoptosis. J Clin Invest 123, 3074-3083 (2013).

9. T. Ura, K. Okuda, M. Shimada, Developments in Viral Vector-Based Vaccines. Vaccines (Basel) 2, 624-641 (2014).

10. Y. C. Chang, V. Nizet, The interplay between Siglecs and sialylated pathogens. Glycobiology 24, 818-825 (2014).

11. A. F. Carlin et al., Molecular mimicry of host sialylated glycans allows a bacterial pathogen to engage neutrophil Siglec-9 and dampen the innate immune response. Blood 113, 3333-3336 (2009).

12. E. Vimr, C. Lichtensteiger, To sialylate, or not to sialylate: that is the question. Trends Microbiol 10, 254-257 (2002).

13. B. Khatua, K. Bhattacharya, C. Mandal, Sialoglycoproteins adsorbed by Pseudomonas aeruginosa facilitate their survival by impeding neutrophil extracellular trap through siglec-9. J Leukoc Biol 91, 641-655 (2012).

14. L. Freire-de-Lima, L. M. Fonseca, T. Oeltmann, L. Mendonca-Previato, J. O. Previato, The trans-sialidase, the major Trypanosoma cruzi virulence factor: Three decades of studies. Glycobiology 25, 1142-1149 (2015).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An isolated polynucleotide comprising: (a) a nucleotide sequence encoding a protein comprising an amino acid sequence encoding a mature Mycobacterium tuberculosis protein selected from AG85A, AG85B, and Ag85C, wherein the protein does not comprise a N-glycosylation consensus sequon, or (b) the full complement of the nucleotide sequence of (a).

2. The isolated polynucleotide of claim 1, wherein (i) the protein encoded by the polynucleotide and the amino acid sequence of SEQ ID NO:1 have at least 80% identity, wherein at least one amino acid of the N-linked glycosylation site NNT at positions 202-204 comprises at least one substitution mutation, (ii) the protein encoded by the polynucleotide and the amino acid sequence of SEQ ID NO:2 have at least 80% identity, wherein at least one amino acid of each of the N-linked glycosylation sites NNS at positions 31-33, NNT at positions 203-205, NGT at positions 213-215, and NGT at positions 259-261 comprises at least one substitution mutation, or (iii) the protein encoded by the polynucleotide and the amino acid sequence of SEQ ID NO:3 have at least 80% identity, wherein at least one amino acid of each of the N-linked glycosylation sites NYT at positions 90-92, NDS at positions 167-169, NNT at positions 201-203, NGT at positions 211-213, NQT at positions 235-237, and NGT at positions 257-259 comprises at least one substitution mutation.

3. The isolated polynucleotide of claim 2 wherein (i) the amino acid at position 202 is substituted, (ii) the amino acids at positions 31, 203, 213, and 259 are substituted, or (iii) the amino acids at positions 167, 201, 211, 235, and 257 are substituted.

4-8. (canceled)

9. The isolated polynucleotide of claim 1, wherein the polynucleotide comprises DNA.

10. The isolated polynucleotide of claim 1, wherein the polynucleotide comprises RNA.

11-14. (canceled)

15. An isolated protein having immunogenic activity, wherein the protein comprises an amino acid sequence, wherein the amino acid sequence and the amino acid sequence of SEQ ID NO:1, 2, or 3 have at least 80% identity, wherein the protein does not comprise a N-glycosylation consensus sequon.

16. The isolated protein of claim 15, wherein the protein is not glycosylated, wherein the protein comprises an amino acid sequence, and wherein the amino acid sequence and the amino acid sequence of SEQ ID NO:1 have at least 80% identity, wherein at least one amino acid of the N-linked glycosylation site NNT at positions 202-204 of SEQ ID NO:1 comprises at least one substitution mutation, (ii) the amino acid sequence and the amino acid sequence of SEQ ID NO:2 have at least 80% identity, wherein at least one amino acid of each of the N-linked glycosylation sites NNS at positions 31-33, NNT at positions 203-205, NGT at positions 213-215, and NGT at positions 259-261 of SEQ ID NO:2 comprises at least one substitution mutation, or (iii) the amino acid sequence and the amino acid sequence of SEQ ID NO:3 have at least 80% identity, wherein at least one amino acid of each of the N-linked glycosylation sites NYT at positions 90-92, NDS at positions 167-169, NNT at positions 201-203, NGT at positions 211-213, NQT at positions 235-237, and NGT at positions 257-259 of SEQ ID NO:3 comprises at least one substitution mutation.

17. The protein of claim 16 wherein (i) the amino acid at position 202 is substituted, (ii) the amino acids at positions 31, 203, 213, and 259 are substituted, or (iii) the amino acids at positions 167, 201, 211, 235, and 257 are substituted.

18-22. (canceled)

23. A genetically modified eukaryotic cell comprising an exogenous polynucleotide, wherein the exogenous polynucleotide is the polynucleotide of claim 9.

24-25. (canceled)

26. A composition comprising the polynucleotide of claim 1 and a pharmaceutically acceptable carrier.

27. (canceled)

28. The composition of claim 26 further comprising an adjuvant.

29. A method for inducing an immune response comprising:
administering to a subject an amount of the composition of claim 26 effective to induce the subject to produce an immune response to the protein encoded by the polynucleotide.

30. The method of claim 29 wherein the immune response comprises a humoral immune response, a cell-mediated immune response, or both a humoral immune response, a cell-mediated immune response.

31. (canceled)

32. A method for treating an infection in a subject, the method comprising:
administering an effective amount of the composition of claim 26 to a subject having or at risk of having an infection caused by Mycobacterium tuberculosis.

33. The method of claim 32 wherein the subject is a mammal.

34. The method of claim 33 wherein the mammal is a human.

35-37. (canceled)

38. A method for inducing an immune response comprising:
administering to a subject an amount of a composition comprising the protein of claim 15 effective to induce the subject to produce an immune response.

39. The method of claim 38 wherein the immune response comprises a humoral immune response, a cell-mediated immune response, or both a humoral immune response, a cell-mediated immune response.

40. A method for treating an infection in a subject, the method comprising:
administering an effective amount of the composition of claim 26 to a subject having or at risk of having an infection caused by Mycobacterium tuberculosis.

41. The method of claim 32 wherein the subject is a mammal.
